(19) United States
(12) Patent Application Publication
(10) Pub. No.: US 2018/0362893 A1
(43) Pub. Date: Dec. 20, 2018

(54) COMPOSITIONS COMPRISING AN ULTRAVIOLET INDICATOR

(71) Applicant: Chemia Corporation, Maryland Heights, MO (US)

(72) Inventors: Alexander N. BRECKWOLDT, Maryland Heights, MO (US); Thomas A. MEYER, Maryland Heights, MO (US); Nicholas VAN REES, Maryland Heights, MO (US)

(21) Appl. No.: 16/060,251

(22) PCT Filed: Dec. 8, 2016
(86) PCT No.: PCT/US2016/065517

§ 371 Date: Jun. 7, 2018

Related U.S. Application Data
(60) Provisional application No. 62/265,766, filed on Dec. 10, 2015.

(51) Int. Cl. C11D 3/40; C11D 3/48; C11D 3/50; A61L 2/28; A61L 2/18; A01N 33/12

(52) U.S. Cl. CPC C11D 3/40; C11D 3/48; C11D 3/50; G01N 21/6447; A61L 2/18; A01N 33/12; A61L 2/28

(57) ABSTRACT

Compositions and methods for evaluating the application of a composition comprising an active compound to a hard surface or skin are provided. Active compounds include an antimicrobial compounds, drug compounds, and consumer product compounds. The compositions comprise at least one UV indicator that will visibly fluoresce upon exposure to a source of UV light. Provided are: (i) concentrate compositions comprising a fragrance and a UV indicator, (ii) concentrate compositions comprising at least one active compound and a UV indicator, (iii) concentrate compositions comprising a fragrance, a UV indicator and at least one active compound, and (iv) diluted and ready to use compositions comprising water, a fragrance, a UV indicator and at least one active compound. Methods for determining if a composition comprising an active compound and a UV indicators has been applied to surface are provided wherein areas of the surface having the composition applied thereto visibly fluoresce upon exposure to UV radiation.

### CROSS REFERENCE TO RELATED APPLICATION

This application claims priority benefit of U.S. Provisional Application Ser. No. 62/265,766 filed on Dec. 10, 2015.

### BACKGROUND OF THE DISCLOSURE

The field of the disclosure relates generally to compositions comprising an ultraviolet indicator and uses thereof to visualize and confirm application of the composition to a surface.

Microbial infection represents a serious continuing problem in human and animal health. This problem is becoming ever more acute due to the proliferation of antibiotic resistant pathogens. Exposure to microbial pathogens can occur in or result from a variety of settings and facilities such as: public facilities; hospitals; consumer product manufacturing facilities; pharmaceutical manufacturing facilities; restaurants; and food processing plants. Controlling and limiting pathogen-related risks presents a major challenge, and failure to adequately disinfect an area may risk the proliferation and spread of pathogens. Further, in topical clinical and topical personal care product use aspects, disinfectant, antibiotic and sunscreen should be applied to a defined skin area. Incomplete application of compositions containing and active agent, such as an antimicrobial, a pharmaceutical, an antibiotic, an antifungal or a sunscreen, could result in ineffective treatment for the intended use.

Cleaning, disinfection, topical personal care, and topical pharmaceutical effectiveness may be enhanced by employing compositions and methods to confirm (validate) application of compositions to a surface in order to assure that all of the areas of a surface to be cleaned have been exposed to the composition.

In some prior art disinfection methods, a UV indicator is applied to a small fraction of an area to be disinfected, a disinfectant composition is applied to the surface and over the UV indicator, the disinfectant composition is wiped off, and the cleaned area is exposed to UV light. The absence of fluorescence is an indication that the UV indicator has been removed and that the area has been disinfected. Problematically, such methods confirm application to only a small portion of the area to be disinfected (i.e., the area to which the UV indicator has been applied) and such methods do not indicate the entirety of the area to which the disinfectant has been applied. Further, problematically, if a user does not apply the UV indicator prior to application of the disinfectant, the absence of fluorescence after cleaning will produce a “false positive” that the area has been cleaned.

A need therefore exists for improved compositions and methods to confirm that a useful composition has been applied to a designated surface.

### BRIEF DESCRIPTION

In some aspects of the disclosure, a composition comprising a solution of a fragrance and a UV indicator is provided. The weight ratio of the fragrance to the dye is from about 1:1 to about 200:1, from about 1:1 to about 150:1, from about 10:1 to about 150:1, about 10:1 to about 50:1, form about 10:1 to about 30:1, from about 50:1 to about 150:1, or from about 75:1 to about 125:1.

In some other aspects of the disclosure, a disinfectant composition is provided. The composition comprises an antimicrobial compound selected from quaternary ammonium compounds, phenolics, iodophors, chlorine compounds, alcohols, ortho-phthalaldehyde, and combinations thereof. The composition further comprises an aqueous carrier, a non-aqueous carrier, or a combination thereof. The composition further comprises a pre-blend comprising (i) a fragrance selected from an ester, an alcohol, a linear terpene, a cyclic terpene, a phenol, a ketone, a lactone, and essential oil, a musk, and combinations thereof and (ii) a UV indicator dye. The concentration of the fragrance in the pre-blend is 1 wt. % to about 99 wt. %, from about 5 wt. % to about 99 wt. %, from about 25 wt. % to about 99 wt. %, from about 50 wt. % to about 99 wt. %, from about 75 wt. % to about 99 wt. %, from about 90 wt. % to about 99 wt. %, or from about 95 wt. % to about 99 wt. %. The weight ratio of the fragrance to the UV indicator dye is from about 10:1 to about 150:1, about 10:1 to about 50:1, form about 10:1 to about 30:1, from about 50:1 to about 150:1, or from about 75:1 to about 125:1, and the concentration of the fragrance in the disinfectant composition is from about 0.01 wt. % to about 5 wt. %, from about 0.1 wt. % to about 3 wt. %, from about 0.1 wt. % to about 1.5 wt. %, or from about 0.1 wt. % to about 1 wt. %.

In some other aspects of the disclosure, a disinfectant composition is provided. The composition comprises an antimicrobial compound is selected from quaternary ammonium compounds, phenolics, iodophors, chlorine compounds, alcohols, ortho-phthalaldehyde, and combinations thereof. The composition further comprises a UV indicator dye. The composition further comprises a fragrance selected from an ester, an alcohol, a linear terpene, a cyclic terpene, a phenol, a ketone, a lactone, and essential oil, a musk, and combinations thereof. The composition further comprises a carrier selected from an aqueous carrier, an anhydrous carrier, and a combination thereof. The concentration of the UV indicator dye is from about 0.005 wt. % to about 0.5 wt. %, from about 0.01 wt. % to about 0.3 wt. %, from about 0.01 wt. % to 0.1 about wt. %, or from about 0.01 wt. % to about 0.08 wt. %.

In some other aspects of the disclosure, a composition is provided comprising: an active compound; a UV indicator dye; a fragrance selected from an ester, an alcohol, a linear terpene, a cyclic terpene, a phenol, a ketone, a lactone, and essential oil, a musk, and combinations thereof and a carrier selected from an aqueous carrier, an anhydrous carrier, and a combination thereof. The concentration of the UV indicator dye is from about 0.005 wt. % to about 0.5 wt. %, from about 0.01 wt. % to about 0.3 wt. %, from about 0.01 wt. % to 0.1 about wt. %, or from about 0.01 wt. % to about 0.08 wt. %.

In some other aspects of the disclosure, a method for determining if a composition comprising an active compound has been applied to surface is provided. The method comprises identifying an area of the surface to which the composition is to be applied; applying the composition to the surface area; and exposing the surface area to a source of UV radiation. Areas of the surface having the composition applied thereto visibly fluoresce.

### DETAILED DESCRIPTION

The disclosure relates generally to aqueous and/or non-aqueous compositions for application to a hard surface or skin, wherein the compositions further comprise a fragrance and an ultraviolet (“UV”) indicator (dye) that will fluoresce upon exposure to ultraviolet radiation. Examples of compositions include, without limitation, soaps, cleaners, sanitizers, lotions, and pharmaceuticals. The compositions are useful for visualization and confirmation of complete and homogeneous application to areas to which the composition is intended to been applied.

In some aspects of the disclosure, the compositions are cleaning and/or disinfectant (antimicrobial) compositions comprising a UV indicator for use on hard surfaces in institutional and industrial settings, for instance and without limitation, hospitals, consumer product manufacturing facilities, pharmaceutical manufacturing facilities, restaurants, schools and food processing plants.

In some other aspects of the disclosure, the compositions are topical pharmaceuticals and/or topical personal care compositions comprising a UV indicator. Examples of topically applied active agents include drugs, antifungals, antibiotics, soaps, alcoholic hand sanitizers and sunscreens.

In some other aspects of the disclosure, the UV indicator compound is dissolved in an aqueous or non-aqueous liquid cleaning and/or disinfectant concentrate to form a pre-blend solution suitable for subsequent formulation in a finished product.

In some other aspects, the fragrance and the UV indicator are formulated directly into a finished product. Finished products within the scope of the present disclosure include, without limitation, automotive cleaners and disinfectants, topical pharmaceuticals and topical consumer products.

In some aspects of the disclosure, the UV indicator compound is dissolved in a liquid fragrance to form a pre-blend solution suitable for subsequent formulation in a finished product.

### Fragrances

Fragrances within the scope of the present disclosure are not narrowly limited and include fragrances approved for use in consumer goods. Published listings of approved fragrances are available. One such listing is published by the International Fragrance Association (“IFRA”) and includes a listing of listing of about 3000 materials classified by CAS Number and a corresponding synonym that were reported in 2011 as fragrance compounds. This database is accessible through IFRA at http://www.ifraorg.org/en-us/ingredients and is incorporated by reference herein. The IFRA fragrance database is not inclusive and other fragrances are in use, or may be developed, that fall within the scope of the present disclosure.

Fragrance chemical classes within the scope of the present disclosure include, but are not limited to, alcohols, esters, ethers, aldehydes, lactones, ketones, acetates, oxides, linear terpenes, phenols, musks, essential oils, cyclic terpenes and benzoids (comprising benzene, naphthalene and/or phenolic moieties), and combinations of at least two chemical classes. Some fragrances within the scope of the present disclosure comprise a combination of chemical classes. For instance, some banana fragrances comprise a combination of at least esters, aldehydes, ketones and lactones. In some aspects, the fragrance may be selected from aliphatic hydrocarbons, terpene hydrocarbons, aromatic hydrocarbons, aliphatic alcohols, terpene alcohols, aromatic alcohols, aliphatic ethers, aromatic ethers, aliphatic oxides, terpene oxides, aliphatic aldehydes, terpene aldehydes, hydrogenated aromatic aldehydes, thioaldehydes, aromatic aldehydes, aliphatic ketones, terpene ketones, hydrogenated aromatic ketones, cyclic ketones, aromatic ketones, acetals, ketals, phenols, phenol ethers, fatty acids, hydrogenated aromatic carboxylic acids, aromatic carboxylic acids, acid amides, aliphatic lactones, macrocyclic lactones, terpene lactones, hydrogenated aromatic lactones, aromatic lactones, aliphatic esters, furan carboxylic esters, alicyclic carboxylic esters, cyclohexylcarboxylic esters, terpene carboxylic esters, aromatic carboxylic esters, nitromusks, nitriles, amines, pyridines, quinolines, pyrroles, and indoles, and combinations of at least two fragrance types.

Examples of fragrances within the scope of the disclosure include, but are not limited to: C₆-C₁₂ aldehydes, ambrox, anisaldehyde, acetal R, acetophenone, acetylcedrene, adoxal, allylamyl glycolate, allyl cyclohexanepropionate, α-damascene, β-damascone, δ-damascone, ambrettolide, ambroxan, amylcinnamic aldehyde, amylcinnamic aldehyde dimethylacetal, amyl valerianate, amyl salicylate, isoamyl acetate, isoamyl salicylate, aurantiol, acetyl eugenol, bacdanol, benzophenone, benzyl acetate, benzyl alcohol, benzyl salicylate, bergamyl acetate, bornyl acetate, butyl butyrate, p-t-butylcyclohexanol, p-t-butylcyclohexyl acetate, o-t-butylcyclohexanol, benzaldehyde, benzyl formate, caryophyllene, cashmerane, carvone, cedramber, cedryl acetate, cedrol, celestolide, cinnamic alcohol, cinnamic aldehyde, cis-jasmone, citral, citral dimethyl acetal, citrasal, citronellal, citronellol, citronellyl acetate, citronellyl formate, citronellyl nitrile, coumarin, cyclaset, cyclamen aldehyde, cyclaprop, caron, coumarin, cinnamyl acetate, γ-C₆-C₁₃ lactone, dimethylbenzylcarbinol, dihydrojasmon, dihydrolinalool, dihydromyrcenol, dimetol, dimyrcetol, diphenyl oxide, elemi gum, ethyl vanillin, eugenol, fruitate, fenchyl alcohol, phenylethyl phenylacetate, galaxolide, γ-C₆-C₁₃ lactone, geraniol, geranyl acetate, geranyl formate, geranyl nitrile, hedion, helional, heliotropin, cis-3-hexanol, cis-3-hexenyl acetate, cis-3-hexenyl salicylate, hexylcinnamic aldehyde, hexyl salicylate, hyacinth dimethyl acetal, hydrotropic alcohol, hydroxycitronellal, indole, ionone, isobornyl acetate, isocyclocitral, Iso E Super, isoeugenol, isononyl acetate, isobutylquinoline, jasmal, jamolactone, jasmopirane, corvone, ligustoral, lilial, lime oxide, limonene, linalool, linalool oxide, linalyl acetate, lyral, manzanate, myol, menthanyl acetate, menthonate, methyl anthranilate, methyl eugenol, menthol, α-methylionone, β-methylionone, γ-methylionone, methyl isoeugenol, methyl lavender ketone, methyl salicylate, muguet aldehyde, mugol, musk TM-II, musk 781, musk C14, musk T, musk ketone, musk tibetene, musk moskene, myrac aldehyde, methyl phenyl acetate, nerol, neryl acetate, nopyl acetate, nopyl alcohol, neobergamate, oak moss No. 1, orivone, oxyphenylon, p-cresyl methyl ether, pentalide, phenylethyl alcohol, phenylethyl acetate, phenylacetaldehyde, dimethyl acetal, α-pinene, rubafuran, rosephenone, rose oxide, Sandalore, Sandela, Santalex, Santalinol, styralyl acetate, styralyl propionate, terpineol, terpinyl acetate, tetrahydrolinalool, tetrahydrolinalyl acetate, tetrahydrogeraniol, tetrahydrogeranyl acetate, tonalide, traseolide, tripral, thymol, vanillin, veltol (maltol), veramoss, verdox, yara yara, anis oil, bay oil, bois de-rose oil, cananga oil, cardamom oil, cassia oil, cedarwood oil, orange oil, mandarin oil, tangerine oil, basil oil, nutmeg oil, citronella oil, clove oil, coriander oil, elemi oil, eucaryptus oil, fennel oil, galbanum oil, geranium oil, hiba oil, hinoki oil, jasmine oil, lavandin oil, lavender oil, lemon oil, lemongrass oil, lime oil, neroli oil, oak moss oil, ocotea oil, patchouli oil, peppermint oil, perilla oil, petitgrain oil, pine oil, rose roil, rosemary oil, camphor oil, ho leaf oil, clary sage oil, sandalwood oil, spearmint oil, spike lavender oil, star anise oil, thyme oil, tonka bean tincture, turpentine oil, vanilla bean tincture, vetiver oil, bergamot oil, ylang ylang oil, grapefruit oil, yuzu (Citrus Junos Tanaka) oil, benzoin, balsam peru, balsam tolu, tuberose oil, musk tincture, castrium tincture, civet tincture, and ambergris tincture. In some aspects of the disclosure, the fragrance component comprises a combination of a least two fragrances.

In some aspects of the disclosure, it has been discovered that certain fragrance ingredients provide UV indication. In some such aspects, the fragrance may suitably be used for UV indication in the absence of an added UV indicator as described elsewhere herein. In some other aspects of the disclosure, it has been discovered that certain fragrance ingredients may boost the fluorescence (efficacy) of UV indicators as described elsewhere herein. In some particular aspects, the fragrance boosts the fluorescence of s-5 white grains UV indicator. Examples of such fluorescing fragrances are listed in Table 1 below:

TABLE 1
Fluorescing Fragrances

| Fluorescing Fragrance | Class | UV Indicator | UV Booster |
|---|---|---|---|
| Veltol (Maltol) | Ketone | Yes | Yes |
| Benzophenone | Ketone | Yes | Yes |
| Veramoss | Phenol | Yes | Yes |
| Ambrox | Musk | Yes | Yes |
| Elemi Gum | Essential Oil | Yes | Yes |
| Coumarin | Lactone | Yes | Yes |

In some aspects of the disclosure, the fragrances and combinations thereof are oil soluble and are suitable for use in disinfectant compositions comprising a non-aqueous component and/or carrier phase. In some other aspects of the disclosure, the fragrances and combinations thereof are water soluble and are suitable for use in disinfectant compositions comprising an aqueous component and/or carrier phase.

### UV Indicators

UV indicators within the scope of the present disclosure are not narrowly limited and are generally any agent (i) that will visibly fluoresce at the concentration used in the compositions of the present disclosure when exposed to radiation having a wavelength of from about 100 nm to about 600 nm, from about 100 nm to about 500 nm or from about 100 nm to about 400 nm, and (ii) that is stable and soluble in the various compositions of the present disclosure. In some aspects of the disclosure, the UV indicator will fluoresce at longwave UV light (UV-A) of from about 315 nm to about 400 nm, at middlewave UV light (UV-B) of from about 280 nm to about 315 nm and/or at shortwave UV light (UV-C) of from about 200 nm to about 280 nm. As used herein in the context of the UV indicator, stable refers to the capability to maintain UV fluorescing properties after formulation and storage before use sufficient to visibly fluoresce upon use.

UV indicator classes within the scope of the present disclosure include, but are not limited to: acridines, cyanines, xanthenes, pyrenes, benzoxazoles, fluoranthenes, phenanthridines, rhodamines, lactones, and quinines, salts thereof, and combinations thereof. The UV indicator may suitably be oil soluble or water soluble, such as a water soluble salt. Examples of fluorescing ultraviolet indicators within the scope of the present disclosure include, but are not limited to, compounds having fluorescent properties such as those disclosed in U.S. Pat. Nos. 3,941,759, 5,227,487, 5,667,840, 5,798,276, 6,080,450 and 6,461,326, each of which are hereby incorporated by reference. For example, in some aspects, the fluorescing UV indicator may comprise: an acridine dye, such as Acridine Orange and Acridine Yellow; cyanine dye, such as Cy3 and Cy5; xanthene dye, such as Eosin, Fluorescein, and Rhodamine; pyrene dye, such as Alex Fluor™ and AMCA-X; benzoxazole dye, such as Uvitex™; fluoranthene dye; quinine dye; and salts thereof. Examples of water-soluble UV indicators include, but are not limited to, Fluorescein, Fluorescein sodium salt, and Pyranine 10G. Examples of oil-soluble UV indicator include, but are not limited to, Pylam Phosphorescence, LX-10522 Pylakrome White and Pylaklor White Grains S-5.

### Combinations of Fragrance and UV Indicator

In some aspects, the compositions of the present disclosure comprise a fragrance and a UV indicator. In some such aspects, the compositions are a concentrate composition comprising, consisting essentially of, or consisting of the fragrance and the UV indicator. The fragrance-UV indicator concentrate composition may then be used as a pre-blend for formulation with other components, including but not limited to, at least one disinfectant compound, an aqueous or non-aqueous carrier, and at least one surfactant. In some aspects, the compositions are disinfectant compositions comprising the fragrance, the UV indicator, at least one disinfectant compound, an aqueous and/or non-aqueous carrier, and at least one surfactant.

In any of the various aspects of the disclosure, the UV indicator is compatible with the fragrance and other disinfectant compositions components as described elsewhere herein such that compositions within the scope of the present disclosure (i) will visibly fluoresce at the concentration used in the compositions of the present disclosure when exposed to radiation having a wavelength of from about 100 nm to about 600 nm, from about 100 nm to about 500 nm or from about 100 nm to about 400 nm, and (ii) are solutions not comprising an appreciable amount of UV indicator precipitated from solution.

In any of the various aspects, the weight ratio of the fragrance to the UV indicator is about 1:1, about 10:1, about 15:1 about 20:1, about 30:1, about 50:1, about 75:1, about 100:1, about 125:1, about 150:1 or about 200:1, and ranges thereof, such as from about 1:1 to about 200:1, from about 1:1 to about 150:1, from about 10:1 to about 150:1, about 10:1 to about 50:1, form about 10:1 to about 30:1, from about 50:1 to about 150:1, or from about 75:1 to about 125:1. In some aspects, the weight ratio of fragrance to UV indicator is from about 10:1 to about 100:1, from about 15:1 to about 100:1, or from about 20:1 to about 100:1.

In concentrate compositions comprising, consisting essentially of, or consisting of a solution of a fragrance and a UV indicator, the concentration of the fragrance is from about 1 wt. % to about 99 wt. %, from about 5 wt. % to about 99 wt. %, from about 25 wt. % to about 99 wt. %, from about 50 wt. % to about 99 wt. %, from about 75 wt. % to about 99 wt. %, from about 90 wt. % to about 99 wt. %, or from about 95 wt. % to about 99.5 wt. %. In some particular aspects, the fragrance concentration in a concentrate composition is about 90 wt. %, about 91 wt. %, about 92 wt. %, about 93 wt. %, about 94 wt. %, about 95 wt. %, about 96 wt. %, about 97 wt. %, about 98 wt. %, about 99 wt. % or about 99.5 wt. % and the remainder of the composition comprises, consists essentially of, or consists of UV indicator in solution.

In diluted and ready-to-use disinfectant compositions, the fragrance concentration is no more than about 1 wt. %, such as about 0.05 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. % or about 1 wt. %, and ranges thereof, such as from about 0.05 wt. % to about 1 wt. % or from about 0.05 wt. % to about 0.5 wt. %. The UV indicator concentration is suitably based on a ratio of the fragrance concentration as describe elsewhere herein.

In some aspects, the fragrance is selected from an ester, a linear terpene, a cyclic terpene, an alcohol, a ketone and a lactone, and combinations thereof. In some other aspects the fragrance is selected from an ester, a linear terpene, a cyclic terpene, a ketone and a lactone, and combinations thereof. In some further aspects, the UV indicator is selected from an acridine, a cyanine, a xanthene, a pyrene, a benzoxazole, a fluoranthene, a phenanthridine, a rhodamine, a lactone, a quinine, salts thereof, and combinations thereof.

### Active Compound

In any of the various aspects of the disclosure, the active compound is selected from a disinfecting antimicrobial compound for use on a hard surface, a drug compound for topical application to skin, and a consumer product compound for topical application to skin.

Drug compounds within the scope of the disclosure include drugs indicated for treating a skin condition. A non-exclusive list of skin conditions include bacterial infections, fungal infections, dermatitis and eczema, psoriasis, hyper-pigmentation, acne, and lesions. Examples of drugs include corticosteroids (e.g., alclometasone, amcinonide, clobetasol propionate, desonide, desoximetasone, diflorasone diacetate, fluocinolone, fluocinonide, flurandrenolide, halcinonide, halobetasol propionate, and mometasone); antifungals (e.g., amorolfine, butenafine, and naftifine hydrochloride); antibiotics (cefdinir, cefoxtamine, cetrimide, chloroxynol, clarithromycin, dapsone, econazole, gatifloxacin, hexachlorophene, mafenide, metronidazole, moxifloxacin, neomycin, ofloxacin, oxytetracycline, quiniodochlor, retapamulin, telavancin, and vancomycin); hyper-pigmentation treatment compounds (e.g., hydroquinone); anti-acne compounds (e.g., isotretinoin); lesion treatment compounds (e.g., methyl aminolevulinate hydrochloride); non-steroidal dermatitis and eczema treatment compounds (e.g., allantoin, pimecrolimus, resorcinol, and retapamulin). Consumer product compounds and compositions within the scope of the present disclosure include sunscreens containing at least one active component such as avobenzone, homosalate, octisalate, octocrylene and oxybenzone.

Disinfecting compounds and compositions within the scope of the disclosure for use on hard surfaces include antimicrobials that are effective to kill or inhibit the growth of one or more microorganisms such as bacteria, viruses and fungi.

In some aspects of the disclosure, the disinfectant may comprise one or more quaternary ammonium compounds (QAC). Examples of QACs include, but are not limited to, benzalkonium chloride, octyl decyl dimethyl ammonium chloride, didecyl-dimethyl-benzyl ammonium chloride, dioctyl dimethyl ammonium chloride, lauryl dimethyl benzyl ammonium chloride, ethylbenzyl chloride, benzethonium chloride, benzyldimethylstearyl ammonium chloride, cetalkonium chloride, cetrimide, cetylpyridinium chloride, cetyltrimethylammonium bromide, dialkyl dimethyl ammonium chloride, N-alkyl dimethyl ethylbenzyl ammonium chloride, and alkyl (e.g., 50% C₁₄, 40% C₁₂ and 10% C₁₆) dimethylbenzyl ammonium chloride. QACs are generally effective when applied at a concentration of from about 0.01 wt. % to about 0.1 wt. % or from about 0.01 wt. % to about 0.05 wt. %, such as about 0.02 wt. %.

In some other aspects of the disclosure, the disinfectant may comprise one or more phenolic compounds. Examples of phenolics include, but are not limited to, triclosan, chloroxylenol, 2-phenylphenol, chlorocresol, 2-benzyl-4-chlorophenol. Phenols are generally formulated at a concentration of from about 0.1 wt. % to about 3 wt. %.

In some other aspects of the disclosure, the disinfectant may comprise one or more iodophor compounds. Examples of iodophores include, but are not limited to, Betadine® and Povidon®. lodophores are generally formulated at a concentration of from about 0.1 wt. % to about 0.3 wt. %.

In other aspects of the disclosure, the disinfectant may comprise one or more chlorine compounds. Examples of chloride compounds include, but are not limited to, bleach (sodium- or calcium-hypochlorite) and chloramine T (tosylchloramide). Chlorine compounds are generally formulated at a concentration of from about 0.05 wt. % to about 2 wt. %.

In other aspects of the disclosure, the disinfectant may comprise one or more alcohols. Examples of alcohols include, but are not limited to, methanol, ethanol, and isopropanol. Alcohols may be formulated at about 50 vol. %, about 60 vol. %, about 70 vol. %, about 80 vol. % or about 90 vol. %, such as from about 50 vol. % to about 90 vol. % or from about 60 vol. % to about 90 vol. %.

In some other aspects of the disclosure, the disinfectant may comprise ortho-phthalaldehyde (“OPA”). OPA may be formulated at from about 0.2 wt. % to about 1 wt. %, such as from about 0.4 wt. % to about 0.6 wt. %.

Some disinfectant compositions of the present disclosure may comprise a combination of at least two disinfectant selected from QACs, phenolics, iodophors, chlorine compounds, alcohols and OPA.

In some aspects of the disclosure, the UV indicator may be dissolved in a non-aqueous disinfectant concentrate composition to form a pre-blend. In some such aspects, the disinfectant concentrate composition may comprise one or more QACs as described elsewhere herein at a total concentration of from about 1 wt. % to about 35 wt. %, from about 5 wt. % to about 30 wt. %, from about 10 wt. % to about 30 wt. %, or from about 15 wt. % to about 25 wt. %.

The disinfectant compositions of the present disclosure may be allowed to dry after application wherein a disinfectant residue remains and provides antimicrobial action over an extended period of time, such as until the disinfectant residue is removed or until the disinfectant degrades.

### Surfactants

The disinfectant compositions may comprises one or more surfactants selected from anionic surfactants, cationic surfactants, amphoteric surfactants, lipophilic non-ionic surfactants and hydrophilic non-ionic surfactants.

Anionic surfactants within the scope of the present disclosure include, but are not limited to: soap bases, sodium laurate, and sodium palmitate; higher alkyl sulfuric ester salts, such as sodium laurylsulfate and potassium laurylsulfate; alkyl ether sulfuric ester salts, such as triethanolamine POE laurylsulfate and sodium POE laurylsulfate; N-acylsarcosinic acids, such as sodium lauroylsarconinate, higher fatty acid amidosulfonates, such as sodium N-myristoyl-N-methyltaurine, sodium palm oil fatty acid methyltauride, and sodium laurylmethyltauride; phosphoric ester salts, such as sodium POE oleyl ether phosphate and POE stearyl ether phosphate; sulfosuccinates, such as sodium di-2-ethylhexylsulfosuccinate, and sodium lauryl polypropylene glycol sulfosuccinate; alkylbenzenesulfonates, such as sodium linear dodecylbenzenesulfonate, triethanolamine dodecylbenzenesulfonate, and linear dodecylbenzenesulfonic acid; N-acylglutamates, such as monosodium N-lauroylglutamate, disodium N-stearoylglutamate, monosodium N-myristoyl-L-glutamate; higher fatty acid ester sulfuric ester salts, such as sodium hydrogenated coconut oil fatty acid glycerol sulfate; sulfated oils, such as Turkey red oil; POE alkyl ether carboxylic acids, POE alkyl allyl, ether carboxylates, α-olefinsulfonates, higher fatty acid ester sulfonates, secondary alcohol sulfuric ester salts, higher fatty acid alkylolamide sulfuric ester salts, sodium lauroyl monoethanolamide succinate, ditriethanolamine N-palmitoylaspartate, and sodium casein.

Cationic surfactants within the scope of the present disclosure include, but are not limited to: stearyltrimethylammonium chloride and lauryltrimethylammonium chloride; dialkyldimethylammonium salts, such as distearyldimethylammonium chloride, alkylpyridinium salts, such as poly-N,N′-dimethyl-3,5-methylenepiperidinium chloride; alkyl quaternary ammonium salts, alkyldimethylbenzylammonium salts, alkylisoquinolinium salts, dialkylmorpholinium salts, POE alkylamines, alkylamine salts, polyamine fatty acid derivatives, amyl alcohol fatty acid derivatives, benzalkonium chloride, and benzethonium chloride.

Amphoteric surfactants within the scope of the present disclosure include, but are not limited to alkyl betaines, amide betaines, sulfobetaines; and amine oxides. Some examples of include sodium 2-undecyl-N,N,N-(hydroxyethylcarboxymethyl)-2-imidazoline and disodium 2-cocoyl-2-imidazolinium hydroxide-1-carboxyethyloxy; and 2-heptadecyl-N-carboxy methyl-N-hydroxy ethylimidazolinium betaine.

Lipophilic non-ionic surfactants within the scope of the present disclosure include, but are not limited to: sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, and diglycerol sorbitan tetra-2-ethylhexylate; glycerol or polyglycerol fatty acids esters, such as glycerol mono-cotton seed oil fatty acid ester, glycerol monoerucate, glycerol sesquioleate, glycerol monostearate, glycerol α,α′-oleate pyroglutamate, and glycerol monostearate maleate; propylene glycol fatty acid esters, such as propylene glycol monostearate; hydrogenated castor oil derivatives, and glycerol alkyl ethers.

Hydrophilic non-ionic surfactants within the scope of the present disclosure include, but are not limited to: POE sorbitan monooleate, POE sorbitan monostearate, POE sorbitan monooleate, and POE sorbitan tetraoleate; POE sorbitol fatty acid esters, such as POE sorbitol monolaurate, POE sorbitol monooleate, POE sorbitol pentaoleate, and POE sorbitol monostearate; POE glycerol fatty acid esters, such as POE glycerol monostearate, POE glycerol monoisostearate, and POE glycerol triisostearate; POE fatty acid esters, such as POE monooleate, POE distearate, POE monodioleate, and ethylene glycol distearate; POE alkyl ethers, such as POE lauryl ether, POE oleyl ether, POE stearyl ether, POE behenyl ether, POE-2-octyldodecyl ether, and POE-cholestanol ether; POE alkyl phenyl ethers, such as POE octyl phenyl ether, POE nonyl phenyl ether, and POE dinonylphenyl ether; Pluronic type surfactants, such as Pluronic; POE/POP alkyl ethers, such as POE/POP cetyl ether, POE/POP 2-decyltetradecyl ether, POE/POP monobutyl ether, POE/POP hydrogenated lanolin, and POE/POP glycerol ether; tetraPOE/tetraPOP ethylenediamine condensates, such as Tetronic; POE castor oil or hydrogenated castor oil derivatives, such as POE castor oil, POE hydrogenated castor oil, POE hydrogenated castor oil monoisostearate, POE hydrogenated castor oil triisostearate, POE hydrogenated castor oil monopyroglutamate monoisostearate, and POE hydrogenated castor oil maleate; POE beeswax lanolin derivatives, such as POE sorbitol beeswax; alkanolamides, such as coconut oil fatty acid diethanolamide, lauric acid monoethanolamide, and fatty acid isopropanolamides; POE propylene glycol fatty acid esters, POE alkylamines, POE fatty acid amides, sucrose fatty acid esters, POE nonylphenyl formaldehyde condensates, alkylethoxydimethylamine oxides, and trioleyl phosphate.

Mixtures of two or more of anionic surfactants, cationic surfactants, amphoteric surfactants, lipophilic non-ionic surfactants and hydrophilic non-ionic surfactants are within the scope of the present disclosure.

In any of the various aspects of the disclosure, the surfactant concentration is about 0.5 wt. %, about 1 wt. %, about 3 wt. %, about 5 wt. %, about 10 wt. %, about 15 wt. %, about 20 wt. % or about 25 wt. %, and ranges thereof, such as from about 0.5 wt. % to about 25 wt. %, from about 1 wt. % to about 25 wt. %, from about 3 wt. % to about 20 wt. %, from about 3 wt. % to about 15 wt. % or from about 5 wt. % to about 10 wt. %.

### Optional Components

In any of the various aspects of the disclosure, the compositions may further comprise one or more inert components and/or enhancing components. Such components include, for instance and without limitation: chelators/sequestrants such as EDTA, tetra- and tri-sodium EDTA, tripotassium EDTA; detergents; and organic acids such as acetic acid, benzoic acid, formic acid, citric acid, lactic acid, malic acid, glutaric acid, propionic acid, and combinations thereof.

### Concentrates

The fragrance and UV indicator may suitably be formulated in a concentrate composition further comprising at least one disinfecting antimicrobial compound for application to a hard surface, at least one drug compound for topical application, or at least one consumer product compound for topical application as described elsewhere herein and further comprising a carrier solvent.

In some disinfectant aspects of the disclosure for application to a hard surface, the disinfectant concentrate is essentially anhydrous (organic) and the carrier is suitably, without limitation, selected from alkanes (e.g., hexane and/or cyclohexane), alcohols (e.g., methanol, ethanol, i-propanol, n-propanol and/or isobutanol), glycols (e.g., ethylene glycol, polyethylene glycol, propylene glycol and/or polypropylene glycol), and mixtures thereof. In some other aspects, the disinfectant concentrate is essentially aqueous. In some other aspects, the disinfectant concentrate carrier comprises water and at least one organic solvent. In some other aspects, the concentrate composition may further comprise at least one surfactant described elsewhere herein. In some aspects, the disinfectant concentrate is a colorless solution, in some aspects, the disinfectant concentrate is a slightly colored solution (such as light yellow appearance), in other aspects, the disinfectant concentrate is a hazy colorless or colored solution. In some aspects, the disinfectant concentrate may comprise a dye that transmits light in the visible spectrum thereby coloring the composition.

In some topical application aspects, the fragrance and UV indicators carrier is suitably formulated with the active substance in a topical formulation as a fragrance-UV indicator concentrate or the fragrance and UV indicator may be individually formulated. Sunscreens may be in the form of anhydrous compositions and water-oil emulsions, and typically comprise components into which the fragrance and dye may suitably be formulated, such as esters comprising a long chain fatty acid (e.g., lauryl laurate, ceteraryl behenate and myristyl myristate), long chain alcohols (e.g., cetyl alcohol, behenyl alcohol and stearyl alcohol), waxes (e.g., beeswax, paraffin and lanolin), petrolatum, surfactants, vegetable oil (e.g., olive oil, coconut oil, sesame oil, almond oil and peanut oil), polyols (e.g., glycerin), glycols (e.g. polyethylene glycol), and water. Topical drug products may be in the form of anhydrous compositions (ointments) and water-oil emulsions, and typically comprise components into which the fragrance and dye may be formulated, such as hydrocarbon base (e.g., paraffin, beeswax, petrolatum, microcrystalline wax, lanolin and cresine), mineral oil, vegetable oil (e.g., olive oil, coconut oil, sesame oil, almond oil and peanut oil), glycols (e.g., polyethylene glycol), water and surfactants. Incorporation of the fragrance and the UV indicator into topical formulations may be done by those skilled in the art without undue experimentation based on the solubility of the fragrance and dye in the components of the topical formulation and using mixing and dissolution methods known in the art.

Exemplary non-limiting ranges for disinfectant concentrate compositions of the disclosure are described in Table 2 below where values are in wt. %:

TABLE 2

| Component | 1^{st} Range | 2^{nd} Range | 3^{rd} Range | 4^{th} Range |
|---|---|---|---|---|
| Antimicrobial | 1 to 35 | 5 to 30 | 10 to 30 | 15 to 25 |
| Carrier | 50 to 90 | 50 to 80 | 55 to 75 | 60 to 70 |
| Fragrance | 0.01 to 5 | 0.1 to 3 | 0.1 to 1.5 | 0.1 to 1 |
| UV indicator | 0.01 to 0.5 | 0.01 to 0.3 | 0.01 to 0.1 | 0.02 to 0.08 |
| Surfactant | 1 to 25 | 3 to 20 | 3 to 15 | 5 to 10 |

One exemplary disinfectant concentrate composition comprises from about 20 wt. % to about 25 wt. % total QAC comprising one or more QAC species; from about 60 wt. % to about 70 wt. % carrier; from about 0.1 wt. % to about 1 wt. % fragrance; from about 0.01 wt. % to about 1 wt. % of a compatible UV indicator; and from about 5 wt. % to about 10 wt. % total surfactant comprising one or more surfactant species.

Disinfectant concentrates may be prepared by mixing methods known to those skilled in the art such as using an agitated vessel and/or using in-line mixers.

### Aqueous Compositions

The fragrance and UV indicator may suitably be formulated in an aqueous composition with at least one antimicrobial compound for application to a hard surface, at least one drug compound for topical application, or at least one consumer product compound for topical application as described elsewhere herein. In some other aspects, the aqueous composition, such as a disinfectant composition, may further comprise at least one surfactant described elsewhere herein. In some such aspects, the aqueous composition may be formed by diluting an active concentrate as described elsewhere herein, such as a disinfectant concentrate, with water to form a disinfectant composition suitable for use on hard surfaces or skin. In some other aspects, the aqueous composition, such as a disinfectant composition, may be directly formulated as a ready-to-use formulation suitable for use on hard surfaces or skin. In some aspects, the aqueous compositions may comprise a dye that transmits light in the visible spectrum thereby coloring the composition.

Exemplary non-limiting ranges for aqueous disinfectant compositions of the disclosure are described in Table 3 below where values are in wt. %:

TABLE 3

| Component | 1^{st} Range | 2^{nd} Range | 3^{rd} Range | 4^{th} Range |
|---|---|---|---|---|
| Antimicrobial | 0.05 to 5 | 0.1 to 3 | 0.1 to 2 | 0.1 to 1 |
| Carrier | 85 to 98 | 85 to 95 | 88 to 95 | 90 to 95 |
| Fragrance | 0.005 to 2 | 0.01 to 2 | 0.05 to 1.5 | 0.1 to 1 |
| UV indicator | 0.005 to 0.5 | 0.01 to 0.3 | 0.01 to 0.1 | 0.01 to 0.08 |
| Surfactant | 0.05 to 5 | 0.1 to 5 | 0.1 to 2 | 0.1 to 1 |

One exemplary aqueous disinfectant composition comprises from about 0.2 wt. % to about 0.5 wt. % total QAC comprising one or more QAC species; from about 90 wt. % to about 95 wt. % carrier; from about 0.1 wt. % to about 1 wt. % fragrance; from about 0.01 wt. % to about 0.08 wt. % of a compatible UV indicator; and from about 0.1 wt. % to about 0.5 wt. % total surfactant comprising one or more surfactant species.

Aqueous disinfectant compositions may be prepared by mixing methods known to those skilled in the art such as using an agitated vessel and/or using in-line mixers.

### Methods

The compositions of the present disclosure may suitably be used according to methods or protocols for visualization and confirmation of application of the compositions to areas to which the composition is intended to been applied.

In some aspects, a cleaning protocol may specify that a disinfectant composition is to be applied to a designated area such as a floor, work surface, process equipment, or the like. Accordingly, compositions of the present invention may be applied to the designated area by methods known in the art such as by spraying, swabbing, mopping, sponging, etc. The applied composition may then be exposed to a source of UV light wherein the applied composition will visibly fluoresce. Based on visible fluorescence, disinfectant application to a surface may be verified. Areas designated to be disinfected and to which the disinfectant composition was not applied may be identified and subjected to a subsequent disinfectant application and visualization confirmation of application. In some aspects, UV visualization may be done after the applied disinfected composition has dried.

In some other aspects, a protocol for topical application of a drug a consumer product may specify that the drug/product be applied to a designated area of skin, such as an area of skin indicated for treatment by a drug or an area of skin indicated for application of a consumer product. Accordingly, drug and/or consumer product compositions comprising a fragrance and a drug of the present disclosure may applied to the designated area by methods known in the art such as by spraying or direct application. The skin may then be exposed to a source of UV light wherein the applied drug/consumer product will visibly fluoresce. Based on visible fluorescence, application to the designated skin area may be verified. Designated skin areas not having applied drug/consumer product may be identified and subjected to a subsequent application and visualization confirmation of application.

### EXAMPLES

### Example 1

The solubility of mixture of the UV indicators Pylam Phosporescence, LX-10522 Pylakrome White and Pylaklor White Grains S-5 at 5 wt. % in Chemia number 60324 anhydrous banana fragrance at 25° C. was evaluated. A visual determination of solubility was made. The results are reported in Table 4 below.

TABLE 4

| | | | |
|---|---|---|---|
| Fragrance | #60324 | #60324 | #60324 |
| Fragrance wt. % | 95 | 95 | 95 |
| UV Indicator | Pylam | LX-10522 | Pylaklor White |
| | Phosporescence | Pylakrome White | Grains S-5 |
| UV wt. % | 5 | 5 | 5 |
| Soluble | No | No | Yes |

The Pylam Phosporescence and LX-10522 Pylakrome White were insoluble in the anhydrous fragrance.

### Example 2

The solubility of Pylaklor White Grains S-5 UV indicator was evaluated in difference chemical classes of fragrances at a UV indicator concentration of 5 wt. %. The resulting admixtures were evaluated for UV activity by exposure to UV light (400 nm ±5 nm) using a Rayovac VBUV-B light source and visual observation. The results are reported in Table 5 below where “Exp” refers to experiment number and “Class” refer to the fragrance chemical class.

TABLE 5

| Exp | Fragrance | Class | Soluble | UV activity |
|---|---|---|---|---|
| 1 | Ethyl acetate | Ester | Yes | Yes |
| 2 | Linalool | Linear terpene | Yes | Yes |
| 3 | D'Limonene | Cyclic terpene | Yes | Yes |
| 4 | Cis-3-hexanol | Alcohol | Yes | Yes |
| 5 | Hexyl cinnamic | Aldehyde | Yes | No |
| | aldehyde | | | |
| 6 | Methyl | Ketone | Yes | Yes |
| | dihydrojasmonate | | | |
| 7 | Gamma-decalactone | Lactone | Yes | Yes |

The solution of hexyl cinnamic aldehyde fragrance changed color and Pylaklor White Grains S-5 UV indicator was not UV active and appears to absorb UV light.

The solution of cis-3-hexanol and Pylaklor White Grains S-5 UV indicator changed from a straw color to a yellow color.

### Example 3

The solubility of Pylaklor White Grains S-5 UV indicator was evaluated in three different aldehyde fragrances at a UV indicator concentration of 5 wt. %. The resulting admixtures were evaluated for UV activity by exposure to UV light (400 nm±5 nm) using a Rayovac VBUV-B light source and visual observation. The results are reported in Table 6 below where “Exp” refers to experiment number and “Class” refer to the fragrance chemical class.

TABLE 6

| Exp | Fragrance | Soluble | UV activity |
|---|---|---|---|
| 1 | Amyl cinnamic aldehyde | Yes | No |
| 2 | Cinnamic aldehyde | Yes | No |
| 3 | Aldehyde C-10 (decanal) | Yes | Yes |

### Example 4

The solubility of a mixture of the UV indicators Pylam Phosporescence, LX-10522 Pylakrome White and Pylaklor White Grains S-5 at 1 wt. % in Lonza HSW-256 quaternary detergent blend concentrate comprising about 9 wt. % benzyl-C₁₂₋₁₆ alkyldimethyl chlorides, about 6.7 wt. % decyldimethyloctyl ammonium chloride, about 4 wt. % didecyldimethyl ammonium chloride, about 2.7 wt. % dimethyldioctyl ammonium chloride, and a non-aqueous carrier was evaluated. The results are reported in Table 7 below.

TABLE 7

| | | | |
|---|---|---|---|
| HWS-256 wt. % | 99 | 99 | 99 |
| UV Indicator | Pylam | LX-10522 | Pylaklor White |
| | Phosporescence | Pylakrome White | Grains S-5 |
| UV wt. % | 1 | 1 | 1 |
| Soluble | No | No | Yes |

Pylam Phosporescence was insoluble and formed an unstable emulsion wherein UV indicator particles precipitated from solution. LX-10522 Pylakrome White formed a stable milky emulsion but that remained opaque throughout testing. Pylaklor White Grains S-5 was soluble in the HWS-256 concentrate and produced a clear and slightly yellow solution.

### Example 5

Applied disinfectant compositions of the present disclosure were evaluated for fluorescence.

In a first experiment, one volume of the solution of Pylaklor White Grains S-5 and HWS-256 concentrate prepared in Example 4 was diluted with 256 volumes of water to form a disinfectant solution suitable for commercial use. There was no color change in the resulting solution. The diluted disinfectant concentrate composition, comprising about 0.05 wt. % Pylaklor White Grains S-5 UV indicator in solution, was applied to a hard surface and allowed to dry. The dried applied disinfectant composition was evaluated for fluorescence using a Rayovac VBUV-B light source. Areas having applied disinfectant clearly fluoresced whereas areas not having applied disinfectant did not fluoresce.

In a second experiment, a fragrance-UV indicator blend comprising 5 wt. % Pylaklor White Grains S-5 UV indicator dye and 95 wt. % banana fragrance was added directly to Lonza HSW-256 quaternary detergent blend concentrate followed by dilution with water to a final UV indicator solution concentration of about 0.05 wt. %. The diluted dye-concentrate was then applied to a hard surface. The applied composition was evaluated for fluorescence using a Rayovac VBUV-B light source and areas having the composition applied thereto clearly fluoresced whereas areas not having the composition applied thereto did not fluoresce.

In a third experiment, a disinfectant concentrate-Pylaklor White Grains S-5 UV indicator solution as prepared in Example 4 (i.e., dye added directly to Lonza HWS-256 to a concentration of 1 wt. % dye) was diluted with water to a final UV indicator solution concentration of about 0.05 wt. % and was applied to a hard surface. The applied composition was evaluated for fluorescence using a Rayovac VBUV-B light source and areas having the composition applied thereto clearly fluoresced whereas areas not having the composition applied thereto did not fluoresce.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A composition comprising a solution of a fragrance and a UV indicator dye wherein:
a. the weight ratio of the fragrance to the dye is from about 1:1 to about 200:1; and
b. the UV indicator dye is stable.

2. The composition of claim 1 wherein the fragrance is selected from an ester, an alcohol, a linear terpene, a cyclic terpene, a ketone and a lactone.

3. The composition of claim 1 further comprising from about 70 wt. % to about 90 wt. % of a non-aqueous carrier.

4. The composition of claim 1 further comprising from about 0.2 wt. % to about 30 wt. % of an aqueous carrier.

5. The composition of claim 1 further comprising at least one antimicrobial compound wherein:
a. the antimicrobial compound is selected from quaternary ammonium compounds, phenolics, iodophors, chlorine compounds, alcohols, ortho-phthalaldehyde, and combinations thereof; and
b. the antimicrobial compound concentration is from about 1 wt. % to about 35 wt. %.

6. The composition of claim 1 wherein the concentration of the fragrance is 75 wt. % to about 99 wt. %.

7. The composition of claim 1 wherein:
a. the fragrance is selected from an ester, a linear terpene, a cyclic terpene, a ketone, a lactone, a phenol, an essential oil, and a musk; and
b. the weight ratio of the fragrance to the UV indicator dye is from about 20:1 to about 100:1.

8. The composition of claim 1 consisting essentially of fragrance and UV indicator dye.

9. The composition of claim 1 wherein the fragrance boosts the fluorescence of the UV indicator.

10. A disinfectant composition comprising:
a. an antimicrobial compound selected from quaternary ammonium compounds, phenolics, iodophors, chlorine compounds, alcohols, ortho-phthalaldehyde, and combinations thereof;
b. an aqueous carrier, a non-aqueous carrier, or a combination thereof; and
c. a pre-blend comprising (i) a fragrance selected from an ester, a linear terpene, a cyclic terpene, an alcohol, a ketone, a lactone and (ii) a UV indicator dye, wherein the concentration of the fragrance in the pre-blend is from about 90 wt. % to about 99 wt. % and wherein the weight ratio of the fragrance to the UV indicator dye is from about 10:1 to about 150:1,
wherein the concentration of the fragrance in the disinfectant composition is from about 0.01 wt. % to about 5 wt. %, and
wherein the UV indicator dye is stable.

11. The composition of claim 10 wherein the antimicrobial compound concentration is from about 0.05 wt. % to about 5 wt. %.

12. The composition of claim 10 wherein the antimicrobial compound is a quaternary ammonium compound.

13. The composition of claim 10 wherein the fragrance boosts the fluorescence of the UV indicator.

14-19. (canceled)

20. A composition comprising:
a. an active compound;
b. a UV indicator dye;
c. a fragrance selected from at least one of an ester, a linear terpene, a cyclic terpene, an alcohol, a ketone, a lactone, a phenol, an essential oil, and a musk; and
d. a carrier selected from an aqueous carrier, an anhydrous carrier, and a combination thereof,
wherein the concentration of the UV indicator dye is from about 0.005 wt. % to about 0.5 wt. %, and wherein the UV indicator dye is stable.

21. The composition of claim 20 wherein the active compound is selected from an antimicrobial compound, a topical drug compound, and a topical consumer product compound.

22. The composition of claim 20 wherein the fragrance boosts the fluorescence of the UV indicator.

23. The composition of claim 20 wherein the weight ratio of the fragrance to the UV indicator dye is from about 10:1 to about 150:1.

24. A method for determining if a composition comprising an active compound has been applied to surface, the method comprising in order:
a. identifying an area of the surface to which the composition is to be applied;
b. applying the composition of claim 20 to the surface area; and
c. exposing the surface area to a source of UV radiation,
wherein areas of the surface having the composition applied thereto visibly fluoresce.

25. The method of claim 24 wherein the active compound is selected from an antimicrobial compound, a drug compound, and a consumer product compound.

26-28. (canceled)

29. The method of claim 24 wherein the fragrance boosts the fluorescence of the UV indicator.
